(19) United States
(12) Patent Application Publication
(10) Pub. No.: US 2025/0095813 A1
(43) Pub. Date: Mar. 20, 2025

(54) COMPUTER-IMPLEMENTED METHOD AND SYSTEM FOR AUTOMATIC CREATION OF A MULTIMEDIA RADIOLOGICAL REPORT AND COMPUTER PROGRAM PRODUCT

(71) Applicant: Siemens Healthineers AG, Forchheim (DE)

(72) Inventors: Sebastian KRUEGER, Erlangen (DE); Stefan THESEN, Dormitz (DE)

(73) Assignee: Siemens Healthineers AG, Forchheim (DE)

(21) Appl. No.: 18/887,204

(22) Filed: Sep. 17, 2024

(30) Foreign Application Priority Data
Sep. 20, 2023 (DE) 10 2023 209 127.5

(51) Int. Cl. G16H 15/00; G16H 10/60

(52) U.S. Cl. CPC G16H 15/00; G16H 10/60

(57) ABSTRACT

One or more example embodiments relates to a method and a system for automatic creation of a multimedia radiological report on a patient, having: a medical information database which has written medical reports on patients; and a data processing unit which is configured: to create a multimedia report script based on the provided written medical report making use of a large language model; to provide selected data to which the written medical report on the patient and/or the created multimedia report script relate; to create a visual medical report on the patient based on the provided data and the created multimedia report script; and which is configured to combine automatically the created visual medical report and audio data for creating the multimedia radiological report on the patient.

### CROSS-REFERENCE TO RELATED APPLICATION(S)

The present application claims priority under 35 U.S.C. § 119 to German Patent Application No. 10 2023 209 127.5, filed Sep. 20, 2023, the entire contents of which are incorporated herein by reference.

### FIELD

One or more example embodiments relates to a method for automatic creation of a multimedia radiological report. One or more example embodiments further relates to a corresponding system and an electronic assembly and a computer program product.

### RELATED ART

Written medical reports with regard to the health status of a patient represent an important result of a medical workflow. The information contained therein is of interest to a plurality of user groups. A referring and/or transferring physician, for example, an orthopedic specialist, refers a patient, in respect of an ailment symptom or a body region, to a radiologist for radiological investigation and for generating corresponding medical images. The radiologist generates the medical images and, based thereon, produces a written medical report formulated in specialist medical language.

However, patients who are usually not medically educated typically have difficulty in understanding written medical reports drawn up by the radiologist. On the other hand, although clinical personnel, referring physicians and other persons involved in the further treatment of the patient are medically trained, they are however only interested in specific information and/or an efficient comprehensibility.

The generation of information material in addition to the written medical report, in order to impart particular information, represents a labor-intensive activity. With rationalized workflows (for example in the domain of the radiologist, image-generation by way of 3D techniques) this additional activity represents a significant workload.

Automatically generated (cinematic) reports can simplify the workflows in the medical environment. For example, rendered images (for example with cinematic rendering) have proved to be advantageous for later operations in the workflows in the domain of imaging, for example, for referring physician communication or in the preparation of an operation on the patient.

Furthermore, animations and videos can imitate interactions and thereby help to enhance spatial understanding and depth perception. It has further been found that spoken language making use of lay terms in conjunction with images make medical matters more readily comprehensible to the patient and also for medically trained personnel. Using different available information sources, it is possible to create a medical report specifically for different recipients and user groups.

Conventionally, the generation of an audio-visual multimedia radiological report takes place manually. For this purpose, 3D technicians are tasked with generating specific images, image sequences and/or additional information (for example measurements) as a service for radiologists and/or referring physicians. The generation of the audio-visual patient communication represents a semi-manual activity for the radiologist and is based upon the written report that the radiologist has already generated. Therein, suitable images must be manually selected by the radiologist or, if suitable images are lacking, must be generated. In addition, it is necessary to create an associated speech output. Additionally, information required for the generation of an audio-visual multimedia radiological report must be laboriously drawn from corresponding sources (data stores, teaching books, etc.).

### SUMMARY

Against this background, one or more example embodiments creates a multimedia radiological report in a rapid and reliable fashion.

### BRIEF DESCRIPTION OF THE DRAWINGS

One or more example embodiments will now be described in greater detail by reference to the schematic figures in the drawings, in which:

FIG. 1 shows a flow diagram to illustrate a possible embodiment of the method for automatic creation of a multimedia radiological report on a patient;

FIG. 2 shows a schematic diagram for explaining the method according to one or more example embodiments for the automatic creation of a multimedia radiological report on a patient;

FIG. 3 shows a schematic block diagram for representing a possible embodiment of the system for automatic creation of a multimedia radiological report (MRB) on a patient;

FIG. 4 shows a JSON script which is used in an exemplary embodiment of the method according to one or more example embodiments.

The accompanying drawings are intended to impart further understanding of the embodiments of the invention. They illustrate embodiments and serve, in relation to the description, to elucidate the principles and concepts of the invention. Other embodiments and many of the stated advantages are disclosed with reference to the drawings. The elements of the drawings are not necessarily shown in correct scale to one another.

### DETAILED DESCRIPTION

Provision is made for:

A computer-implemented method for automatic creation of a multimedia radiological report on a patient, having the steps: providing a written medical report on the patient; creating a multimedia report script on the basis of the provided written medical report, making use of a large language model (LLM); providing data to which the written medical report on the patient relates and/or the created multimedia report script relates; creating a visual medical report on the patient on the basis of the provided data and the created multimedia report script; and combining the visual medical report and audio data for creating the multimedia radiological report on the patient.

A system for automatic creation of a multimedia radiological report on a patient, in particular making use of a method according to one or more example embodiments, with a medical information database which has written medical reports on patients, and with a data processing unit which is configured: to create a multimedia report script on the basis of the provided written medical report, making use of a large language model (LLM); to provide data to which the written medical report on the patient and/or the created multimedia report script relate; to create a visual medical report on the patient on the basis of the provided data and the created multimedia report script; and which is configured to combine the created visual medical report and audio data for creating the multimedia radiological report on the patient.

A computer program product with a stored program code which comprises program commands for carrying out a method for automatic creation of a multimedia radiological report on a patient, having the following steps: providing a written medical report on the patient; creating a multimedia report script on the basis of the provided written medical report, making use of a large language model; providing data to which the written medical report on the patient and/or the created multimedia report script relate; creating a visual medical report on the patient on the basis of the provided data and the created multimedia report script; and combining the visual medical report and audio data for creating the multimedia radiological report on the patient.

Advantageous embodiments and developments are disclosed in the subclaims and in the description making reference to the figures in the drawing.

In a possible embodiment of the method, the multimedia report script has a layout of the multimedia radiological report and a list of keyframes.

In a possible embodiment of the method, the keyframes of the created multimedia report script have, at least by way of a region to be investigated, in particular, an anatomical structure to be investigated, a visualization type, visualization parameters, text information, available external data and time information. By this means, the method can be used flexibly for a large number of applications.

In a possible embodiment of the method, the keyframe text information of the created multimedia report script is automatically converted, making use of a further large language model (LLM), into lay terms. This offers the advantage that a patient can more easily understand the multimedia radiological report generated for him and fewer queries to the referring physician arise.

In a possible embodiment of the method, for providing keyframe audio data, the lay terms are synthesized in spoken language. This has the advantage that the automatic generation of the multimedia radiological report takes place reliably and rapidly.

In a possible embodiment of the method, the synthesized keyframe audio data is combined with the visual medical report to create the multimedia radiological report. This offers the advantage of a fully automatic generation of the multimedia radiological report without any manual intervention.

In a possible embodiment of the method, the synthesized keyframe audio data is synchronized with the visual medical report, making use of the keyframe time information contained in the multimedia report script, to create the multimedia radiological report on the patient. This increases the comprehensibility for the patient of the created multimedia radiological report.

In a possible embodiment of the method, the created multimedia radiological report on the patient is output via a patient portal. In this way, the patient receives rapid access to his multimedia radiological report in a reliable manner.

The above embodiments and developments can be combined with one another as desired, wherever useful. Further possible configurations, developments and implementations of the invention also include not explicitly mentioned combinations of features of the invention described above or in the following in relation to the exemplary embodiments. In particular, a person skilled in the art would also draw upon individual aspects as improvements or enhancements of the respective basic form of the present invention.

Independent of the grammatical term usage, individuals with male, female or other gender identities are included within the term.

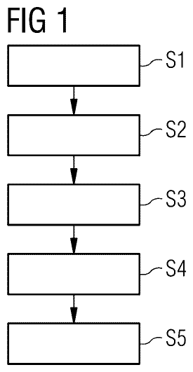

As the flow diagram in FIG. 1 shows, a computer-implemented method for automatic creation of a multimedia radiological report (MRB) on a patient in a possible embodiment comprises a plurality of steps:

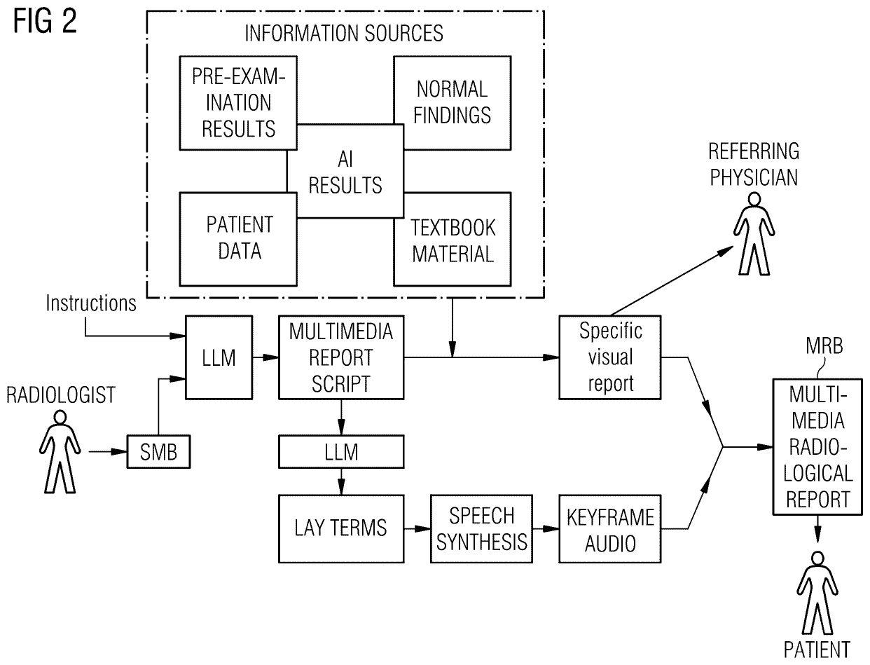

In a first step S1, a written medical report (SMB) on the patient is provided. This written medical report SMB is dictated, for example, by a radiologist and comprises text as shown in FIG. 2. The written medical report SMB is placed in intermediate storage and fed to a large language model LLM. Depending upon the application case, different sizes of language model can be utilized, for example GPT4. The large language model LLM is trained via an unsupervised machine learning process. Advantageously, the large language model LLM additionally receives instructions (guidelines/prompts/ground truth) which train it specifically for the creation of a multimedia report script, i.e. in a supervised training process, the large language model LLM used is specifically trained for this.

In a further step S2, a multimedia report script is created automatically on the basis of the provided written medical report, making use of the trained large language model (LLM). The multimedia report script is generated and/or written automatically by the large language model LLM on a case-by-case basis. For example, the script is generated in JSON by the large language model LLM used. JSON (JavaScript Object Notation) defines a data format in which information such as objects, arrays and other variables can be stored in a readable form.

In a possible embodiment of the method, the multimedia report script specifies a layout of the multimedia radiological report and a list of keyframes. Keyframes comprise key scenes of an animation which each have a sequence of images. In one possible embodiment of the method, the keyframes of the created multimedia report script relate to at least one region to be investigated, in particular, an anatomical structure of the patient that is to be investigated. Furthermore, for each keyframe, a visualization type, visualization parameter, text information, available external data and time information can be specified in the script.

In one possible embodiment of the method, the keyframe text information of the created multimedia report script is automatically converted into lay terms making use of the same or a further large language model (LLM), i.e. into terms or words which are intelligible to a lay person, in particular a patient. In one possible embodiment of the method, for providing keyframe audio data, the lay terms are synthesized via speech synthesis into spoken language, as shown in FIG. 2.

A large language model (LLM) is used in step S2 in order, from a provided written medical report SMB, to generate a script/formula. In one possible embodiment, this script/formula contains a layout of the report (for example individual film, document) and a list of the “keyframes”. These keyframes can be specified, for example, by way of an associated anatomical region/organ/anatomical landmark/etc. Furthermore, the manner of the visualization (2D, 3D, 2D+t, 3D+t, original images, MPR, cinematic) can be specified in the script. Furthermore, different visualization parameters (for example cinematic rendering description), a description of how which diagnoses/regions of interest are visualized (emphasizing, pointers, markings, etc.), textual information (for example findings), additional external data (for example earlier images, teaching book text/illustration, visualization of a normal case) and a time plan in relation to the final report (video) can be given in the script.

In a further step S3, selected data is provided to which the written medical report on the patient and/or the created multimedia report script relates. For example, the radiologist can explicitly make reference in his written medical report SMB to particular data, in particular, to particular medical images, in particular, in that he explicitly mentions the images, giving an image name or image ID in the text. In many cases, however, the radiologist will not refer in the text of the written medical report (SMB) to particular images, but will rather describe an ailment scenario therein or a diagnosis (for example herniated spinal disk at vertebra L5) in general. An image AI can automatically select suitable medical images therefrom. For example, the image AI can select medical images of the patient in the region of vertebra L5.

The selected data, in particular, image data, can be provided by way of different information sources, as shown schematically in FIG. 2. The information sources comprise, for example, pre-examination results from the patient under investigation, normal findings, AI results, patient data from the patient and textbook material (for example, anatomical drawings in standard medical works or teaching books). This information and/or data is found in different databases which are accessible via a data network for a data processing unit DVE of the system according to one or more example embodiments.

With the aid of the previously created script/formula, the required patient data and external data can be selected and collected.

In a further step S4, a visual medical report on the patient is created on the basis of the provided selected data and the multimedia report script created by way of the large language model LLM.

The visual medical report is generated on the basis of the selected data and the script/formula. The result can be passed on with just the usual tools (DICOM), for example, to a referring person. The referring person is the person who refers the patient to the radiologist to generate the written medical report.

In a further step S5, the visual medical report created in step S4 with audio data to create the multimedia radiological report MRB on the patient is combined and/or merged.

In a possible embodiment of the method, the synthesized keyframe audio data is combined with the visual medical report to create the multimedia radiological report. In a possible embodiment of the method, the synthesized keyframe audio data is synchronized with the visual medical report, making use of the keyframe time information contained in the multimedia report script, to create the multimedia radiological report on the patient. The visual report is combined with the synthetic audio information and is matched with the temporal description in the script.

In a possible embodiment of the method, in a further step, the multimedia radiological report on the patient created in S5 is output to the patient via a patient portal. The resultant multimedia video report can be transferred to the patient via an existing patient portal (DICOM-based). Subsequently, the patient has the possibility of discussing the multimedia radiological report MRB with the referring physician.

The method according to one or more example embodiments will now be described in greater detail.

A patient has fallen from a chair. The patient attempted to save himself with his hand on impact.

The associated imaging comprises a left wrist: intraarticular (radiocarpal, radioulnar), distal fracture of the radius with multiple fragments. The carpus and the fragments are displaced by 1 cm dorsally relative to the radial shaft.

The associated diagnosis reads:

Recent, displaced fracture of the distal left radius Classification: xyz

The associated translation of the diagnosis into terms comprehensible to a lay person reads:

“On falling from the ladder, you broke your right wrist. The joint capsules of both bones of the lower arm are affected. In addition, the bone fragments and the wrist itself have been displaced by 1 cm toward the back of the hand.”

The associated screenplay and/or script (with consecutively numbered keyframes) reads:

Animation 8 seconds, from keyframe 1) to 2) (as a visible rotation of the hand imaging)
1) 0 seconds:
a. Hand, left, including wrist
i. View from front (hand inner surface, ventrally)
ii. 3D imaging, bones only, no skin or muscles
iii. Arrows on radial and ulnar joints
b. Text “On falling from the ladder, you broke your right wrist. The joint capsules of both bones of the lower arm are affected.”
2) 8 seconds:
a. Hand, left, including wrist
i. View from side (laterally)
ii. 3D imaging, bones only, no skin or muscles
iii. Arrow to dislocation with text “1 cm”
b. Text “In addition, the bone fragments and the carpus itself are displaced by 1 cm toward the back of the hand.”

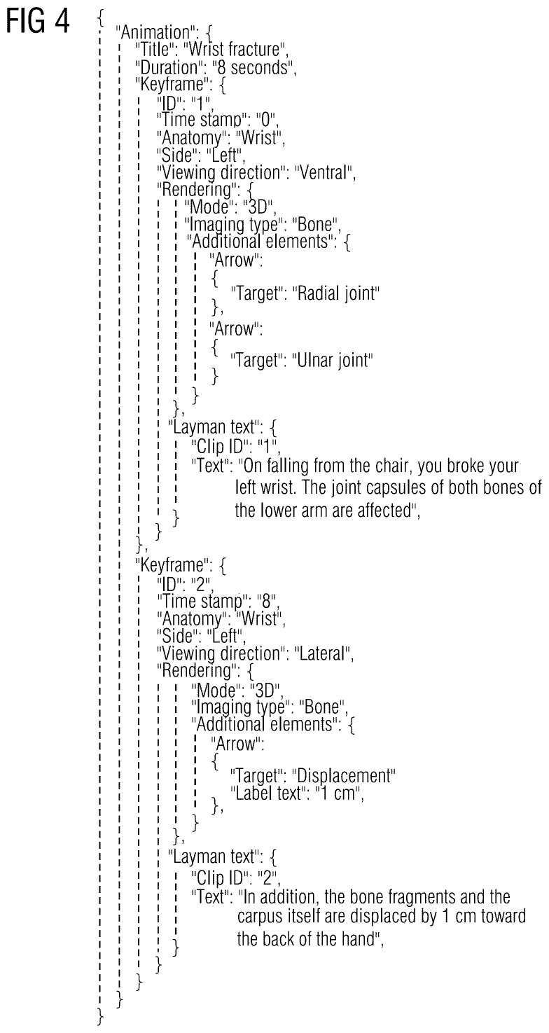

The corresponding JSON script created in the method according to one or more example embodiments is shown in FIG. 4.

In one possible embodiment, the multimedia video report and/or multimedia radiological report MRB can be viewed by a user, for example, the patient, including via an AR or VR headset.

Further embodiment variants are possible. For example, the patient can ask questions acoustically via a microphone, recorded on a further soundtrack, regarding the received multimedia radiological report MRB. This multimedia radiological report as prepared can then be transferred to the referring physician who, in this way, has the opportunity to prepare for the personal consultation with the patient.

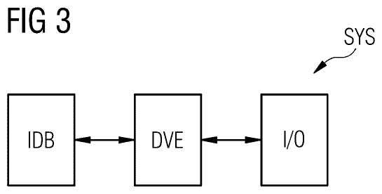

One or more example embodiments further provides a system for automatic creation of a multimedia radiological report on a patient, as shown schematically in FIG. 3.

The system comprises at least one medical information database IDB which has written medical reports on the patient.

The system further has a data processing unit DVE which is configured to create a multimedia report script on the basis of the provided written medical report, making use of a large language model (LLM); to provide data to which the written medical report on the patient and/or the created multimedia report script relates; and to create a visual medical report on the patient on the basis of the provided data and the created multimedia report script.

The data processing unit DVE is further configured for automatically combining the created visual medical report and synthesized audio data for creating the multimedia radiological report MRB on the patient.

The data processing unit DVE has access to a large number of information sources, as shown schematically in FIG. 2.

Furthermore, the system has a user interface I/O (for example a portal) in order to output to the patient the generated multimedia radiological report MRB on the patient. The generated multimedia radiological report MRB is preferably also transferred to a terminal of the referring and/or transferring physician.

The method according to one or more example embodiments and the system according to one or more example embodiments permit an automatic generation of scripts/formulae that describe medical diagnoses at a meta-level.

The method according to one or more example embodiments and the system according to one or more example embodiments further enable the automatic creation of image material, in particular, in conjunction with (photorealistic) medical volume rendering. The method according to one or more example embodiments and the system according to one or more example embodiments implement a complete pipeline (report->audio-visual information).

The method according to one or more example embodiments and the system according to one or more example embodiments also enable an automatic generation of written/spoken language, the automatic generation of image material on the basis of a written report, and offer the possibility of generating audiovisual information therefrom on the basis of report data.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units various that perform operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store programs, computer program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible language), markup (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); t computer-readable medium is therefore considered tangible and non-transitory. Non-limiting examples of the non-transitory computer-readable medium include, but are not limited to, rewriteable non-volatile memory devices (including, for example memory flash devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for automatic creation of a multimedia radiological report on a patient, the method comprising:
providing a written medical report on the patient;
creating a multimedia report script based on the provided written medical report using a large language model;
providing data related to at least one of the written medical report on the patient or the created multimedia report script;
creating a visual medical report on the patient based on the provided data and the created multimedia report script; and
combining the visual medical report and audio data to create the multimedia radiological report on the patient.

2. The method of claim 1, wherein the multimedia report script includes a layout of the multimedia radiological report and a list of keyframes.

3. The method of claim 2, wherein the keyframes of the created multimedia report script includes, at least by way of a region to be investigated, at least one of at least one visualization parameter, text information, available external data or time information.

4. The method of claim 3, wherein the keyframe text information of the created multimedia report script is automatically converted into lay terms using a further large language model.

5. The method of claim 4, wherein the lay terms are synthesized in spoken language for providing keyframe audio data.

6. The method of claim 5, wherein the keyframe audio data is combined with the visual medical report to create the multimedia radiological report.

7. The method of claim 6, wherein the keyframe audio data is synchronized with the visual medical report using the keyframe time information contained in the multimedia report script to create the multimedia radiological report on the patient.

8. The method of claim 1, wherein the created multimedia radiological report on the patient is output via a patient portal.

9. A system for automatically creating a multimedia radiological report on a patient, the system comprising:
a medical information database including written medical reports on patients; and
a data processing unit configured to cause the system to implement the method of claim 1.

10. A non-transitory computer program product with a stored program code which comprises program commands for carrying out a method for automatic creation of a multimedia radiological report on a patient comprising:
providing a written medical report on the patient;
creating a multimedia report script based on the provided written medical report using a large language model;
providing data related to at least one of the written medical report on the patient or the created multimedia report script;
creating a visual medical report on the patient based on the provided data and the created multimedia report script; and
combining the visual medical report and audio data to create the multimedia radiological report on the patient.
